(19) United States
(12) Patent Application Publication
(10) Pub. No.: US 2005/0131309 A1
(43) Pub. Date: Jun. 16, 2005

(54) Blood pressure measuring apparatus

(75) Inventors: Peter Gotz, Greiling (DE); Jorg Ziel, Greiling (DE); Werner Wingender, Greiling (DE); Jochen Von Benthen, Bad Tolz (DE)

Correspondence Address: BURNS DOANE SWECKER & MATHIS L L P, POST OFFICE BOX 1404, ALEXANDRIA, VA, 22313-1404, US

(73) Assignee: ERKA, Kallmeyer Medizintechnik GmbH & Co. KG

(21) Appl. No.: 10/767,612

(22) Filed: Jan. 30, 2004

(30) Foreign Application Priority Data
Jan. 31, 2003 (DE) 103 03 906.6

(51) Int. Cl. A61B 5/02

(52) U.S. Cl. 600/491

(57) ABSTRACT

In connection with a mechanical blood pressure measuring device having a pressure measuring cell with a pressure indicator for measuring the course of pressure and an operating unit with a pressure generating device and a presure regulating device, the pressure measuring cell and the operating unit are releasably connected with each other. The pressure measuring cell and the operating unit can be connected with each other in two positions which are turned by 180° in respect to each other.

### CROSS REFERENCE TO RELATED APPLICATIONS

This application claims priority under 35 U.S.C. §119 to German Application 103 03 906.6 filed in Germany on 31 Jan. 2003, the entire contents of which are hereby incorporated by reference in their entirety.

### BACKGROUND

1. Technical Field

The present invention relates to a mechanical blood pressure measuring apparatus, in particular an apparatus comprising an operating unit for generating and regulating pressure by hand.

2. Description of the Related Art

One type of mechanical blood pressure measuring apparatus in accordance with the prior art comprises a pressure sleeve which for example is fastened to the upper arm by means of a Velcro fastener, and a hand apparatus comprising an operating unit for generating and regulating pressure as well as a measuring cell which comprises a meter and a display for indicating the pressure progression. In order to generate pressure in the sleeve, it is connected to the hand apparatus by one or two flexible tubes and can for example be pumped up by the user, using a pump ball on the operating unit. At the same time as generating pressure in the sleeve, pumping up also generates a pressure in the measuring cell, which is determined by the meter and displayed by the pressure display. In order to regulate the pressure, the hand apparatus comprises for example a valve on the operating unit, said valve being closed while pressure is built up and opened for measuring the blood pressure, such that the pressure in the sleeve and in the measuring cell is slowly and continuously reduced. This requires a very fine adjustability of the valve, in order to be able to regulate the pressure exactly, from which a systole pressure and a diastole pressure can be clearly recognized at a particular point in time.

On one side of the pump ball, a fixed element is provided on the operating unit, such as for instance a scoop which lies in the heel of the hand and against which the pump ball can be pressed using the fingers. When generating pressure, the scoop thus serves as a stabilizing element when handling the blood pressure measuring apparatus. The valve is provided on a side opposite the scoop, i.e. on the other side of the pressure ball, such that the valve can for example be operated using a forefinger and thumb, while the pump ball continues to be held in the heel of the hand together with the scoop. The measuring cell is arranged at a front end of the pump ball and a change in pressure can be read from the display of said measuring cell, while the user holds the pump ball in his/her hand and regulates the valve.

Such a blood pressure measuring apparatus can be operated with one hand. In order to ensure that the blood pressure is measured exactly and reliably, it is necessary to calibrate the meter in the measuring cell at regular intervals. To this end, the blood pressure measuring apparatus is generally sent to the manufacturer or a repairer authorized by the manufacturer, where it is tested and calibrated and sent back again to the user.

The conventional blood pressure measuring apparatuses described have the disadvantage that the arrangement of the scoop, pump ball, valve and pressure display is configured for operation using the right hand. If the apparatus is used by a left-handed person, who also holds the scoop in the heel of the hand, operates the pump ball using the fingers and then regulates the valve using the forefinger and thumb, the apparatus is rotated by 180° with respect to its use by a right-handed person. The pressure display therefore points away from the user, who cannot then read it.

Apparatuses have therefore been developed in which the scoop on the pressure ball can be arranged on the opposite side as compared to an arrangement for right-handed people, such that even a left-handed person can easily press the pump ball against the scoop. However, it is then hardly possible to operate the valve with the same hand, since this is on the same side as the scoop and is not accessible using the fingers. Furthermore, blood pressure measuring apparatuses have been developed which comprise a pressure button for regulating the pressure, instead of a laterally arranged turning valve, said pressure button being arranged at the top on the side of the hand apparatus facing the user, such that it can be swapped from the right hand to the left and operated in the same way, from above. However, such pressure regulators are unfamiliar for a physician or medical assistant and exhibit the disadvantage that fine-tuning the pressure is possible only with difficulty.

A further disadvantage of the conventional blood pressure measuring apparatus is that the entire measuring apparatus is unavailable while the calibration is performed. A second apparatus is therefore required for the user, in order to continue to be able to check the blood pressure regularly.

### SUMMARY OF THE INVENTION

A blood pressure measuring apparatus is disclosed which can easily be adjusted to different users and applications, can be handled flexibly, reduces maintenance costs and reduces or even rules out periods of unavailability.

An exemplary blood pressure measuring apparatus comprises a measuring cell including a meter for measuring a pressure progression and a pressure display, e.g. in the form of a scale sheet, over which a pointer turns in accordance with the prevalent pressure. The blood pressure measuring apparatus further comprises an operating unit comprising a pressure generating device, such as a latex-free pump ball, and a pressure regulating device such as for instance a release valve made, for example, of metal, such as chrome-plated brass. Using the release valve, the pressure progression can be fine-tuned by means of a turning mechanism, such that so-called slip-stick effects can be avoided. The measuring cell and the operating unit can be detachably connected to each other, i.e. in an operationally ready state for measuring the blood pressure, the measuring cell and the operating unit can be detachably connected to each other and form a rigid unit. The connection can, however, be released, such that the measuring cell and the operating unit can be moved against each other. In this way, for example, they can remain connected to each other but be rotated of shifted against each other. The measuring cell and the operating unit can be detachably connected to each other in such a way that they can be completely separated, put together again, and fixedly connected to each other.

By designing the blood pressure measuring apparatus in this way, it is possible to remove the measuring cell from the operating unit and make the measuring cell alone available for maintenance work, such as for instance a prescribed regular calibration. In the event of a complete failure of the measuring cell, i.e. due to irreparable faults in the meter, it is possible to merely exchange the measuring cell for a new, functioning and tested measuring cell and to continue to use the original operating unit. It is also possible to manufacture a measuring cell cost-effectively, such that in can be provided as a sort of disposable cell which can be disposed of and replaced by a new measuring cell as soon as a calibration or repair is necessary. In this sense, the measuring cell forms an exchange unit which ensures that the blood pressure measuring apparatus can be used continuously.

In an exemplary embodiment of the blood pressure measuring apparatus, the measuring cell and the operating unit can be connected to each other in at least two different predetermined positions with respect to each other. A first position of the operating unit with respect to the measuring cell can be suitable for use by a right-handed person and a second position of the operating unit with respect to the measuring cell can be suitable for use by a left-handed person, when looking down on the display of the measuring cell. In the case of an operating unit which for example comprises a cruciform holder comprising four connector ends, the measuring cell can for example be arranged at one end, such that the display is visible to a user, and the pump ball can be attached to the opposing end. By arranging the measuring cell and the pump ball in a line, an axis of the blood pressure measuring apparatus is defined. At the connector ends of the holder, running transverse to this axis, a scoop and a connector for a sleeve tube can be arranged at one of said ends of the holder, for example the right-hand end when looking down on the display, wherein the scoop extends along the pump ball on said right-hand side. On the connector end of the holder opposite this end, i.e. on the left-hand end when looking down on the display of the measuring cell, a release valve can be provided opposite the scoop. Accordingly, in a first position when looking down on the measuring cell, the pump ball is arranged opposite the measuring cell, the release valve on the left-hand side and the scoop or sleeve tube on the right-hand side. In an exemplary embodiment of the blood pressure measuring apparatus, the operating unit is rotated with respect to the measuring cell, in a second position, by 180° relative to the first position described above, along the axis formed by linearly arranging the measuring cell and the pump ball. Accordingly, the measuring cell and the pump ball remain in their position in such a second position. The positions of the scoop and the release valve, however, are swapped, such that the scoop is arranged on a left-hand side of the pump ball and the valve on a right-hand side, when looking down on the display. It is then advantageous if the pump ball is formed rotationally symmetrical with respect to the axis described above, such that it does not change the shape of its arrangement when it is turned. It is of course also conceivable to provide other predetermined positions of the measuring cell and the operating unit with respect to each other, such as for instance positions rotated by 90° about the axis described above. It is also possible to provide more than two different predetermined positions, such as for instance four or eight positions.

The blood pressure measuring apparatus can be easily adapted to different operational requirements due to the possibility of fixedly connecting the measuring cell and the operating unit to each other in different positions, and/or releasing a fixed connection in a particular position and fixedly connecting the measuring cell and the operating unit to each other in another position. In particular, it is possible to adapt the blood pressure measuring apparatus to the particular requirements of right-handed and left-handed people without any problems and without having to sacrifice user-friendliness of the apparatus.

A guiding means can be provided for guiding the measuring cell or the operating unit into the at least two predetermined positions. To this end, for example, a guiding groove or guiding area can be arranged on the measuring cell, onto which the operating unit is placed, for example via a connector end, and moved into the predetermined position along said guiding means. It is also possible to provide, as the guiding means, a first guiding agent on the measuring cell and a second guiding agent on the operating unit, wherein the first and second guiding agents cooperate when the measuring cell and the operating unit are put together. In one embodiment of the invention, the measuring cell comprises a flat cylindrical casing which is provided with an inlet port on its lateral circumferential area, for connecting to the pressure system or operating unit. A support area which is slightly convex in a circumferential direction with respect to the port is provided around the inlet port as a guiding means. For connecting the measuring cell to the operating unit, a connector end can be inserted into the inlet port of the measuring cell. To this end, the connector end can be placed on the support area of the measuring cell and shifted towards the port until it abuts. Other guiding means are also possible, such as for instance a pin on the operating unit which engages with a guiding groove on the measuring cell, or a turning mechanism with different abutting or locking positions, such that the measuring cell and the operating unit are arranged in a predetermined position with respect to each other when they abut or are locked.

In an exemplary embodiment the measuring cell and the operating unit can be connected to each other by a latching, locking or plugging connection, which are formed detachable. To this end, for example, a latching means can be provided on the measuring cell and a latching counter means can be provided on the operating unit. When the measuring cell and the operating unit are guided together by means of the guiding means, the latching means and latching counter means can be guided towards each other until they latch into each other in an end position, i.e. in a predetermined position with respect to each other.

In an exemplary embodiment of the blood pressure measuring apparatus, a removable bolting element is provided for connecting the measuring cell and the operating unit, for easy handling. The measuring cell and the operating unit are thus joined to each other in, a predetermined position with respect to each other and detachably connected to each other with the aid of the bolting element. To this end, latching, locking or plugging means are for example provided on both the measuring cell and the operating unit, with which latching, locking or plugging counter means engage which are arranged on the removable bolting element. It is then advantageous, for example, to provide a number of such means at different positions on the operating unit corresponding to different predetermined positions between the measuring cell and the operating unit. A first latching or locking means can for example be arranged on an upper side on a connector end of the operating unit and a second latching or locking means can be arranged on a lower side opposite said upper side. The operating unit can thus be attached to the measuring cell using the bolting element both in a first position and in a second position turned 180° with respect to said first position. Using the bolting element, the measuring cell and the operating unit are therefore fixedly connected to each other in a selected position. If the bolting element is removed, the measuring cell and the operating unit can be moved against each other and for example moved into another predetermined position or completely separated from each other. The measuring cell can thus be quickly adjusted with respect to the operating unit or quickly exchanged, without another tool being required for this purpose.

An elastic element can be arranged in the connecting region between the measuring cell and the operating unit, wherein both the measuring cell and the operating unit abut said element in the detachably fixed position. This can for example enable a clearance-free connection between the measuring cell and the operating unit, since the measuring cell and the operating unit are pressed against the elastic element, wherein an axial bias can arise between the measuring cell and the operating unit. To this end, an elastic ring, for instance made of rubber or plastic, can for example be inserted in the inlet port of the measuring cell, the connector end of the operating unit abutting said ring once it has been inserted into the port. When the measuring cell is connected to the operating unit, for example using the removable bolting element, the operating unit can be pressed against the ring such that a fixed, biased connection arises between the measuring cell and the operating unit.

In an exemplary embodiment, an identifying means for individualizing the apparatus is provided on the blood pressure measuring apparatus. The allocation of the apparatus to a user can thus be easily and immediately recognized. It is possible, for example, for a left-handed person to immediately find a blood pressure measuring apparatus adapted to his/her requirements among a number of apparatus. The identifying means can be attached with the bolting element. Thus, when adapting the apparatus to a user, the identifying means can be attached at the same time. To this end, the bolting element can comprise a plate-like region and is at least partially formed transparent, such that an identifying means, for example in the form of an inscribed or marked insert, can be arranged between the bolting element and the operating unit or measuring cell. The removable bolting element is then advantageously arranged on the upper side, i.e. the side with the pressure display of the measuring apparatus, such that the identification can always be read from above, in any position of the operating unit with respect to the measuring cell. It is of course also possible to attach the identification to a clearly visible point on the blood pressure measuring apparatus, independently of the connection between the measuring cell and the operating unit.

In the blood pressure measuring apparatus, the release valve for regulating the pressure in the measuring cell comprises a release screw which is advantageously secured by a locking connection on the valve which forms a forcible lock. Via the locking connection, the release screw can be removed and replaced without another tool being required. A split pin, in the form of a spring bow, can for example be used as a locking element, tensed around a cylinder element of the valve onto which the release screw is slid. The split pin engages with a groove on the circumference of the cylinder element and comprises a projection, formed radially inwards, which engages with a hole of a regulating element through a hole in the groove of the cylinder element, such that they are connected to each other, secured against rotating. The release screw is slid over the spring bow, such that a locking connection is formed between the cylinder element and the release screw.

The pump ball, which forms the pressure generating device on the operating unit, comprises a port at one end with a region sliding inwards, via which it is arranged on a sleeve-shaped connector end of the holder of the operating unit. The region sliding inwards thus encloses the connector end air-tight. To this end, the pump ball can be made for example of a plastic material which can be processed in a rotational molding method. As opposed to a dipping method, in which a connecting region sliding outwards is created on the pump ball, in a rotational molding method it is possible to form the pump ball with a connecting region sliding inwards. An inner connecting region enables a stable connection to the holder of the operating unit and the operating unit as a whole can be formed shorter.

Securing the release screw using a clamping element on the valve, and a pump ball comprising a connecting region sliding inwards, form advantageous embodiments of a blood pressure measuring apparatus, which can also be employed independently of the other features of the invention.

### BRIEF DESCRIPTION OF THE DRAWINGS

A blood pressure measuring apparatus is explained in more detail by way of an example embodiment and with the aid of the drawings, wherein:

FIG. 1 shows a perspective, exploded view of an exemplary embodiment of a blood pressure measuring apparatus;

FIG. 2 shows a sectional view through a holder of an operating unit in accordance with an exemplary embodiment;

FIG. 3 shows a view onto a casing part of a measuring cell in accordance with an exemplary embodiment;

FIG. 4 shows a perspective view of a scoop of the operating unit, in accordance with an exemplary embodiment;

FIG. 5 shows a lateral view of a removable bolting element in accordance with an exemplary embodiment; and

FIG. 6 shows a sectional view through a pump ball of an operating unit in accordance with an exemplary embodiment of a blood pressure measuring apparatus.

### DETAILED DESCRIPTION

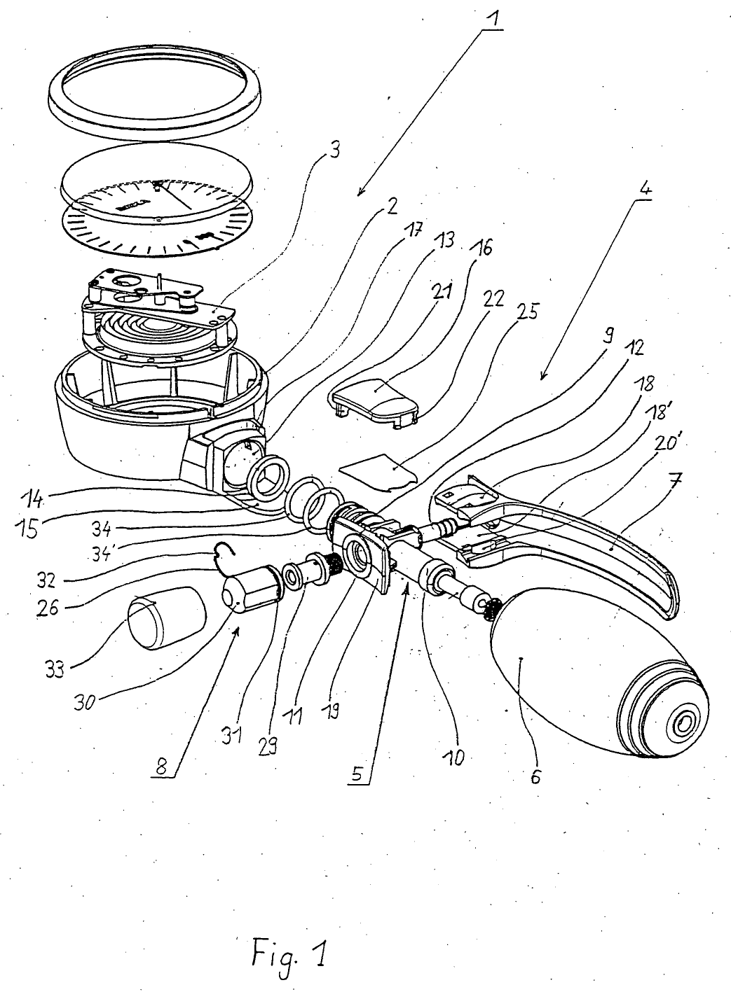

FIG. 1 shows an exploded view comprising the essential individual parts of an exemplary blood pressure measuring apparatus. The blood pressure measuring apparatus comprises a measuring cell 1 including a casing part 2, a meter 3 and a scale, a transparent cover and a sealing ring. The casing part is formed as a cylindrical element having a small height as compared to its diameter, such that a flat cell results. In the circumferential area, the casing part 2 comprises a connector port 13 via which the measuring cell 1 can be connected to a pressure generating device of the blood pressure measuring apparatus. An operating unit 4 of the blood pressure measuring apparatus comprises a holder 5 to which a pressure generating device, comprising a pump ball 6 and a scoop 7, and a pressure regulating device in the form of a release valve 8 are attached.

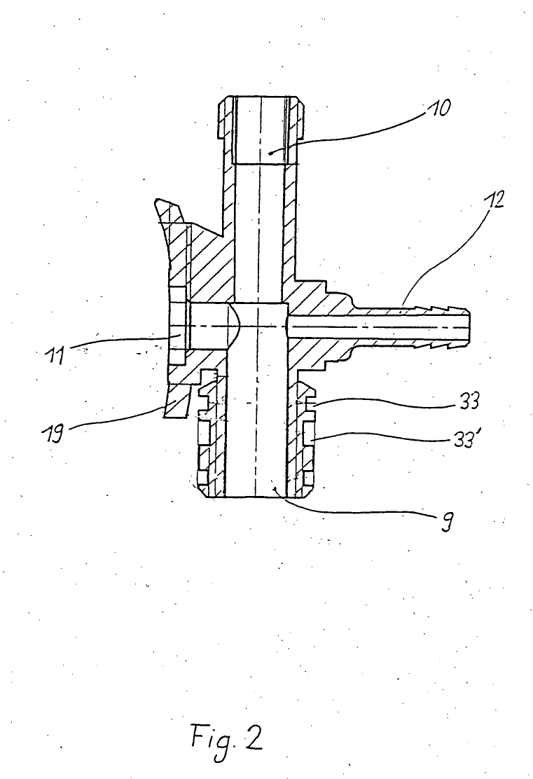

As shown in FIG. 2, the holder 5 is formed as a cruciform support element comprising four connector ends 9, 10, 11 and 12 which are connected to each other in the interior of the holder 5 by a hollow space. The connector ends 9, 10, 11 and 12 exhibit a sort of tubular shape and open outwards. The measuring cell 1 is positioned on the end 9 of the holder by means of the connector port 13 and the pump ball 6 is fastened to the end 10 opposite the end 9. The holder 5 forms a rotational axis of the blood pressure measuring apparatus along the ends 9 and 10, the axis intersecting the circumferential area of the casing part 2 substantially perpendicularly. The opposing ends 11 and 12 of the holder 5 run transverse to the ends 9 and 10. The release valve 8 is positioned on the end 11 and the scoop 7 is positioned on the end 12. The scoop 7 runs approximately perpendicular to the end 12, substantially parallel to the rotational axis of the blood pressure measuring apparatus. The scoop 7 is then bent in such a way that it laterally abuts a surface of the pump ball 6. A flexible tube (not shown) can also be positioned on the end 12 of the holder 5, the tube running to a sleeve attached to an upper arm, for measuring pressure. The hollow space in the interior of the holder 5 establishes a connection between the pump ball 6, the measuring cell 1 and the sleeve, such that these parts are connected to each other in terms of pressure. A pressure can be built up in the sleeve and in the measuring cell 1 using the pump ball 6. The pressure can be regulated using the release valve 8, which is inserted into and seals the end 11 of the holder 5.

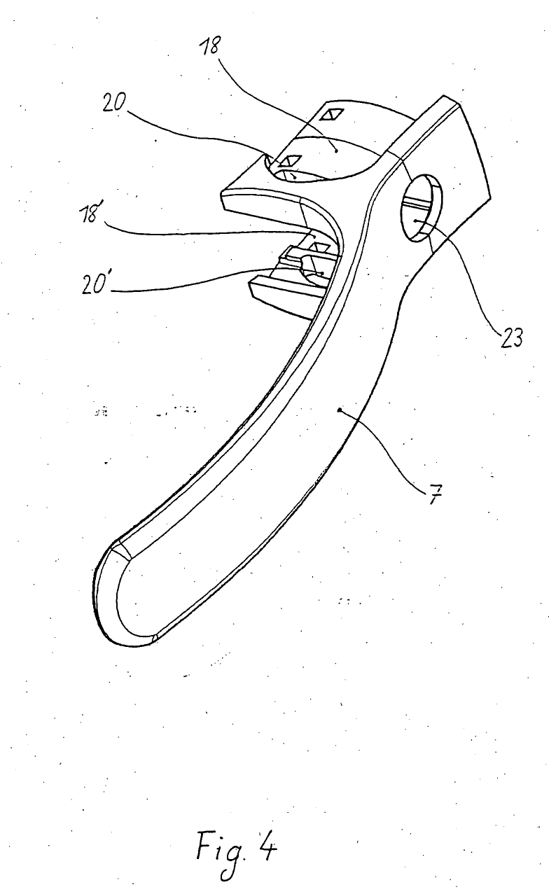

The scoop 7 of the operating unit 4 is formed by two parallel, opposing areas 18 and 18′, as shown in FIG. 4, between which the holder 5 comes to rest, wherein a port 23 is provided in a scoop region connecting the areas 18 and 18′, the end 9 of the holder 5 being guided through said port 23. The end 11 of the holder 5 is surrounded by a plate 19 running perpendicular to the end 11. The areas 18 and 18′ of the scoop 7 surround the holder 5 in such a way that they terminate with the plate 19. The scoop 7, the areas 18 and 18′ and the plate 19 thus form a sort of casing region for the holder 5.

In accordance with an exemplary embodiment, it is possible to attach the operating unit 4 to the measuring cell 1 in various predetermined positions. In a first position, as shown in FIG. 1, the scoop 7 of the operating unit 4 lies on the right-hand side of the pump ball 6 and the release valve 8 lies on the left-hand side. In a second position, the operating unit 4 is turned, for example by 180°, about the rotational axis formed by the ends 9 and 10 of the holder 5, with respect to the measuring cell 1, such that the scoop 7 is arranged on the left-hand side and the release valve 8 on the right-hand side of the pump ball 6.

For connecting the measuring cell 1 to the operating unit 4, the casing part 2 of the measuring cell 1 is positioned onto the end 9 of the holder 5 via its connector port 13. To this end, grooves 33 and 33′ are provided on the end 9 (FIG. 2), onto which O-rings 34 and 34′ can be positioned, to establish a connection between the measuring cell 1 and the operating unit 4. When the end 9 is inserted into the connector port 13, the O-rings 34 and 34′ come to rest in the connector port 13 in such a way that the measuring cell 1 and the operating unit 4 are connected to each other in a positive lock. The O-rings 34 and 34′ thus seal the connection off pressure-tight. When a tensile force is applied, the positive lock is released and the measuring cell 1 can be removed from the operating unit 4. The rings 34 and 34′ thus generate a sort of releasable frictional connection between the measuring cell 1 and the operating unit 4. An elastic element, for example in the form of a rubber ring 14, can be inserted between the casing part 2 and the end 9 of the holder 5. The rubber ring 14 serves to establish a clearance-free connection between the measuring cell 1 and the operating unit 4.

In the region of the base of the casing part 2, a guiding means is formed at the connector port 13, in the form of a tongue 15 protruding perpendicularly from the port. As it approaches the circular connector port 13, the tongue 15 is formed convex around said port. The end 9 of the holder 5 can be guided into the connector port 13 along the tongue 15.

In order to bolt the measuring cell 1 to the operating unit 4, in a selected position with respect to each other, a removable bolting element 16 is provided which comprises a bolting means 21, 22 both for the measuring cell 1 and for the operating unit 4. It is of course also possible to connect the measuring cell 1 and the operating unit 4 to each other directly, without other elements, using a suitable connection. For connecting to the bolting element 16, the casing part 2 of the measuring cell 1 comprises a locking means 17 on a side of the connector port 13′ opposite the tongue 15, i.e. on the side of the display.

Locking means 20 and 20′ are provided in the areas 18 and 18′ of the holder 5. The bolting element 16 is formed partially plate-like and comprises perpendicularly projecting locking counter means 21 and 22 on opposing sides. The locking counter means 21 of the bolting element 16 cooperates with the locking means 17 on the casing part 2 of the measuring cell 1 and the locking counter means 22 cooperates with the locking means 20 or 20′ on an area 18 or 18′, respectively, when the measuring cell 1 and the operating unit 4 are positioned on each other. A locking connection with the area 18 thus corresponds to a first predetermined position between the measuring cell 1 and the operating unit 4, for use of the measuring apparatus by a right-handed person, and a locking connection with the area 18′ corresponds to a second predetermined position, for use of the measuring apparatus by a left-handed person, said second position being turned with respect to the first position by 180° about the axis of the holder 5 formed by the ends 9 and 10. The bolting element 16 engages with both the locking means 17 and the locking means 20 or 20′ and forms a fixed connection between the measuring cell 1 and the operating unit 4 and bolts it in the predetermined position. This presses the operating unit 4 against the rubber ring 14, such that a clearance-free connection is created. In such an arrangement, the tongue 15 comes to rest on the opposite side of the bolting-element 16 on the area 18 or 18′ which is not used to establish the connection. As a whole, therefore, a casing of the blood pressure measuring apparatus is formed from the casing part 2 together with the tongue 15, the bolting element 16, the scoop 7 and the plate 19. The bolting element 16 can be released from the locking connection and removed from the blood pressure measuring apparatus. In this way, the operating unit 4 can be released from the casing part 2 of the measuring cell 1 and for example attached to the casing part 2 in a position turned by 180°.

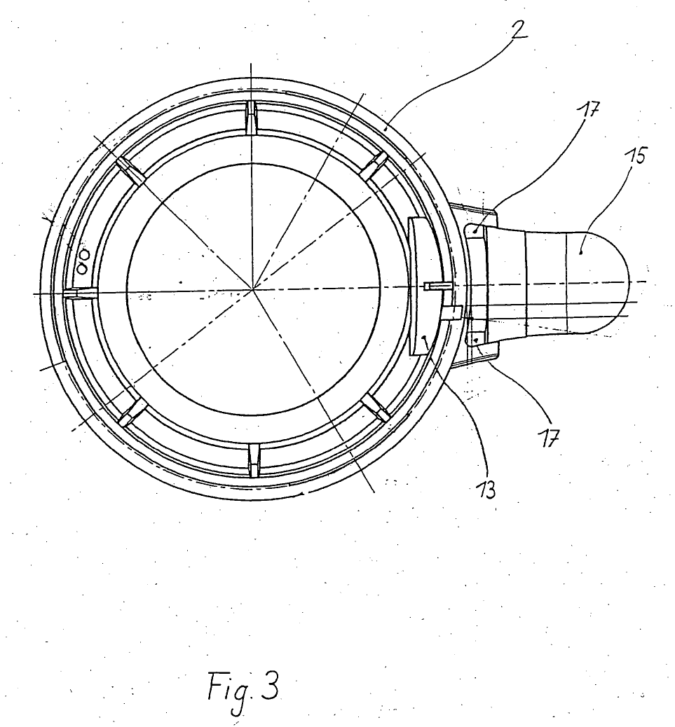

The locking connection of an exemplary embodiment is shown by the individual elements in FIGS. 3, 4 and 5. FIG. 3 shows the casing part 2 together with the locking means 17 in the form of recesses provided on both sides of the connector port 13, perpendicular to the area of the display. FIG. 4 shows the scoop 7 together with the areas 18 and 18′, in each of which a locking means 20 is provided in the form of an elongated cavity.

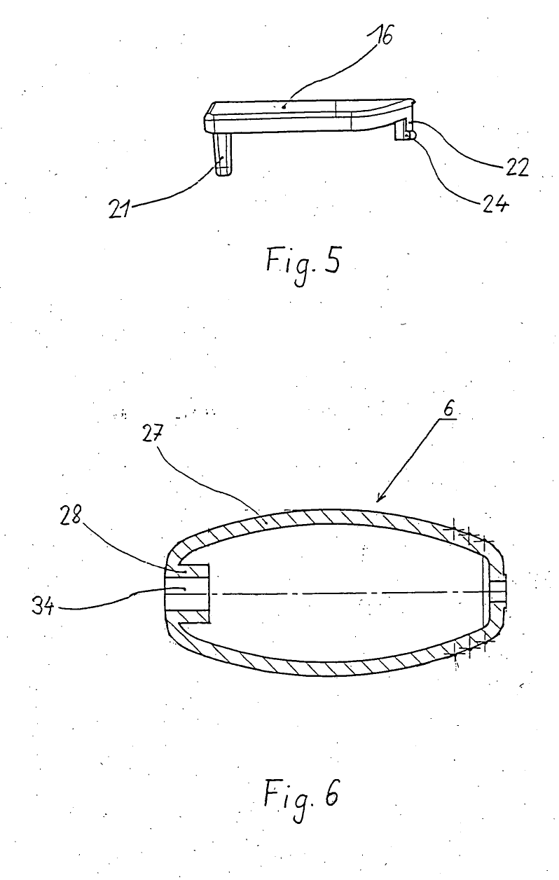

The partially plate-like bolting element 16 is shown in FIG. 5. A locking counter means 21, in the form of two appendages, is provided on both sides at one end of the bolting element 16, projecting perpendicularly from the plate-like area. A locking counter means 22 is arranged on the opposite side of the bolting element 16, the locking counter means 22 having two projections which project in the same direction as the appendages 21. For establishing the connection between the measuring cell 1 and the operating unit 4, the bolting element 16 is inserted via the appendages 21 into the recesses 17 of the casing part 2. A transverse connection between the appendages 21 thus engages with a groove 35 on the end 9 of the holder 5 (see FIG. 2). Via the projections 22, the bolting element 16 is pressed into the cavity 20 or 20′ on an area 18 or 18′ of the scoop 7. This biases the projections 22, such that they lock into the cavity 20 via a bulge 24 on the protruding end of the projections 22. The blood pressure measuring apparatus is then ready for operation. If the measuring cell′ 1 is to be exchanged, the bolting element 16 can be removed, such that the connection between the measuring cell 1 and the operating unit 4 can be released.

An identifying means 25 can be attached to the blood pressure measuring apparatus, to individualize the apparatus for a particular user, by inserting for example a slip of paper 25 between the area 18 or 18′ and the bolting element 16. The name of a user or a colored marking allocated to a particular department can for example be printed on the slip of paper. The bolting element 16 can be formed transparent, such that the identification can be read through the bolting element 16. It is also possible to not form the entire bolting element transparent, but to only provide a window over the area of the identifying means 25. The bolting element 16 thus fulfils the function of identifying the blood pressure measuring apparatus, connecting the measuring cell 1 to the operating unit 4, and forms a casing part of the blood pressure measuring apparatus.

The release valve 8 of the operating unit 4 is pressed into the end 11 of the holder 5 by means of a cylindrical valve collar 29 onto which a knurled screw 30 is positioned, thus creating a pressure-tight connection. Advantageously, a groove 31 with a hole through the wall of the knurled screw may be provided on a circumferential region of the knurled screw 30 near the end 11. A sort of spring bow 26 is inserted in the groove 31 and encompasses the knurled screw 30 due to its bias. The spring bow comprises a projection 32 formed radially inwards, which engages with a recess in the valve collar 29 through the hole in the groove 31 of the knurled screw 30 and thus attaches the knurled screw 30, secured against rotating, with respect to the valve collar 29. A sleeve-like release screw 36 can exhibit a groove on its inner circumference, the groove coming to rest over the spring bow 26 when the release screw 36 is positioned onto the knurled screw 30, such that the release screw 36 is connected to the knurled screw 30 by a locking connection. In this way, the release screw 36 is fixedly connected, for regulating the pressure via the release valve 8, but can be removed from the knurled screw 30, such that the spring bow 26 can be released and the release valve 8 can be disassembled as a whole, without additional tools, for maintenance work.

FIG. 6 shows an oval pump ball 6 with an elastic wall 27 and comprising a port 34 at one end, into which the end 10 of the holder 5 can be inserted. To facilitate assembly and for a shorter design of the operating unit, a wall region 28 surrounding the port 34 is formed sliding inwards into the pump ball. In addition to facilitating assemble, another advantage of the wall region 28 formed sliding inwards is that no regions of the pump ball 6 come to rest between the areas 18 and 18′ of the scoop 7, such that it can be removed from the holder 5 without having to remove other elements of the operating unit 4 from the holder 5.

The invention has been explained by way of exemplary embodiments. It is, however, clear that the essential features can also be fulfilled by a different embodiment of individual elements of the blood pressure measuring apparatus. For example, instead of a locking connection between the measuring cell 1 and the operating unit 4, a screw connection can also be used. Furthermore, it is of course possible to provide more than two predetermined positions of the operating unit 4 with respect to the measuring cell 1, in which the measuring cell 1 and the operating unit 4 can enter into a fixed connection to each other. The scope of the invention is thus not to be restricted, by the examples shown.

It will be appreciated by those skilled in the art that the present invention can be embodied in other specific forms without departing from the spirit or essential characteristics thereof. The presently disclosed embodiments are therefore considered in all respects to be illustrative and not restricted the scope of the invention is indicated by the appended claims rather than the foregoing description and all changes that come within the meaning and range and equivalence thereof are intended to be embraced therein.

1. A hand-operated blood pressure measuring apparatus comprising:
a measuring cell having a pressure display for measuring a pressure progression; and
a hand-operated operating unit having a pressure generating and regulating device, wherein said measuring cell and said operating unit are detachably connected to each other.

2. The hand-operated blood pressure measuring apparatus as set forth in claim 1, wherein a holder end is provided for the measuring cell, said holder end is allocated to the measuring cell, and the measuring cell can be detachably connected to said holder end.

3. The hand-operated blood pressure measuring apparatus as set forth in claim 1, wherein the measuring cell and the operating unit can be connected to each other in at least two different predetermined positions with respect to each other.

4. The hand-operated blood pressure measuring apparatus as set forth in claim 1, wherein the measuring cell and the operating unit can be connected to each other in two positions rotated by 180° with respect to each other.

5. The hand-operated blood pressure measuring apparatus as set forth in claim 3, wherein a guiding means is provided for guiding at least one of the measuring cell and the operating unit into the at least two predetermined positions.

6. The hand-operated blood pressure measuring apparatus as set forth in claim. 1, wherein the measuring cell and the operating unit can be connected to each other by at least one of a latching, a locking and a plugging connection.

7. The hand-operated blood pressure measuring apparatus as set forth in claim 1, wherein the measuring cell and the operating unit are connected to each other by at least one of a removable connecting element and a bolting element.

8. The hand-operated blood pressure measuring apparatus as set forth in claim 7, wherein the measuring cell and the operating unit are bolted to each other using the at least one of the removable connecting elements and the bolting element.

9. The hand-operated blood pressure measuring apparatus as set forth in claim 1, wherein an elastic element is provided between the measuring cell and the operating unit.

10. The hand-operated blood pressure measuring apparatus as set forth in claim 9, wherein said elastic element is arranged pressed between the measuring cell and the operating unit when the measuring cell and the operating unit are connected.

11. The hand-operated blood pressure measuring apparatus as set forth in claim 1, comprising:
an identifying means for individualizing.

12. The hand-operated blood pressure measuring apparatus as set forth in claim 11, wherein said identifying means is attached to at least one of a removable connecting element and a bolting element used to connect the measuring cell and the operating unit.

13. The hand-operated blood pressure measuring apparatus as set forth in claim 7, wherein the removable connecting and bolting element comprise a plate-like region which is at least partially formed transparent.

14. The hand-operated blood pressure measuring apparatus as set forth in claim 13, wherein a connecting means for connecting to the measuring cell and a connecting means for connecting to the operating unit are provided on the at least one connecting element and bolting element.

15. The hand-operated blood pressure measuring apparatus as set forth in claim 1, wherein the measuring cell is provided as an exchange unit.

16. The hand-operated blood pressure measuring apparatus as set forth in claim 1, wherein the pressure generating and regulating device comprises a valve with a release screw secured by a clamping connection on said valve.

17. The hand-operated blood pressure measuring apparatus as set forth in claim 16, wherein said clamping connection is generated by a spring bow.

18. The hand-operated blood pressure measuring apparatus as set forth in claim 1, wherein the pressure generating device comprises a pump ball which comprises a connecting region sliding inwards to a holder of the operating unit.
